(19) United States
(12) Patent Application Publication
(10) Pub. No.: US 2025/0041080 A1
(43) Pub. Date: Feb. 6, 2025

(54) JOINT

(71) Applicant: OTTOBOCK SE & CO. KGAA, Duderstadt (DE)

(72) Inventor: Martin PUSCH, Duderstadt (DE)

(21) Appl. No.: 18/717,146

(22) PCT Filed: Dec. 7, 2022
(86) PCT No.: PCT/EP2022/084858

§ 371 Date: Jun. 6, 2024

(30) Foreign Application Priority Data
Dec. 7, 2021 (DE) 10 2021 132 201.4

(51) Int. Cl. A61F 2/74; A61F 2/64; A61F 2/66

(52) U.S. Cl. CPC A61F 2/744; A61F 2/6607; A61F 2/64; A61F 2/748

(57) ABSTRACT

The invention relates to a joint for an orthopedic device. The joint has a first joint part (38), a second joint part (34), which is pivotally arranged on the first joint part (38) about a pivot axis (36) in a pivoting region, and a hydraulic system with at least one first cylinder (2), which has a first longitudinal axis and is arranged in the second joint part (34), and a first piston (20) which is positioned in the cylinder (2), wherein the first piston (20) is arranged on a first securing point (64) of the first joint part (38) such that the first piston (20) carries out a movement along the first longitudinal axis of the first cylinder (2) and a tilting movement when the first joint part (38) is pivoted relative to the second joint part (34).

The invention relates to a joint for an orthopedic device, the joint having a first joint part, a second joint part, which is arranged on the first joint part such that it can be swivelled about a swivel axis in a swivel range, and a hydraulic system with at least one cylinder, which comprises first longitudinal axis and is arranged in the second joint part, and a first piston that is positioned the cylinder.

Such joints have been known from the prior art for many years and are used in the prior art to fulfil a wide range of requirements. Such a joint can be used, for example, as an ankle joint in a leg prosthesis. In this case, one of the two joint parts is connected to a prosthetic foot or is formed by a prosthetic foot. The respective other joint part is generally designed with a connecting element, such as a pyramid adapter, for connecting a further prosthetic component, such as a lower leg, to this joint part. The joints enables a movement that imitates the ankle movement of the human foot between the two joint parts, e.g. the prosthetic foot and a lower leg. The movement of the two joint parts relative to each other is counteracted by a damping and a force by way of the flow resistance of the hydraulic fluid via a hydraulic system.

Such a hydraulic system usually comprises two hydraulic chambers that are fluidically connected to each other. If the joint is now moved, i.e. the first joint part is swivelled relative to the second joint part about the swivel axis, the hydraulic fluid is directed from one of the two hydraulic chambers into the respective other of the two hydraulic chambers. In the process, hydraulic lines or/or valves, such as a throttle valve used for this purpose, apply a flow resistance, which is perceived as a damping of the movement of the joint.

The two joint parts can now only be swivelled about the swivel axis within a swivel range, i.e. a predetermined angular range. This swivel range is delimited on both sides by an end stop.

Conventionally, the hydraulic chambers are located in a common cylinder and separated from each other by a piston. As an alternative, a separate cylinder can also be provided for each hydraulic chamber, a piston being movably arranged in each cylinder. Regardless of the embodiment used, the fluid is directed from one hydraulic chamber into the other hydraulic chamber by moving the pistons provided. In the prior art, it is important that the movement of the piston follows the shape of the cylinder. The cylinder generally has a longitudinal axis, along which its cross-section is constant. The movement of the piston within the cylinder then follows this longitudinal direction and is composed solely of such a displacement along one direction. Since hydraulic fluid must be prevented from passing between the piston and the inner wall of the cylinder, the piston is usually fitted with a seal on its outer surface, which faces the inner wall of the cylinder. To ensure that it produces a sufficient sealing effect in every position of the piston within the cylinder, in the prior art it is ensured that the orientation of the piston within the cylinder does not change.

The invention thus relates to a joint for an orthopedic device, the joint comprising a first joint part and a second joint part, which is arranged on the first joint part such that it can be swivelled about a swivel axis. The swivel axis may be a fixed swivel axis, which extends along a shaft for example, or a virtual swivel axis. Such a joint also has a hydraulic system, preferably with a first hydraulic chamber, a second hydraulic chamber that is fluidically connected to the first hydraulic chamber by at least one fluid connection, and at least one valve that is configured to open and close the fluid connection. The hydraulic system is arranged and configured such that hydraulic fluid flows from the first hydraulic chamber into the second hydraulic chamber or vice versa when the first joint part is swivelled relative to the second joint part. The invention also relates to a method for setting a starting position of the first joint part relative to the second joint part of such a joint and an orthopedic device that has such a joint.

Within the context of the present invention, an orthopedic device refers in particular to a prosthesis or an orthosis. They are preferably designed for the lower limb and the joint is used as a knee joint, a hip joint or an ankle joint.

Joints according to the preamble, which are referred to as hydraulic joints, have been known from the prior art for many years and can essentially be used in two different ways.

It is particularly, but not exclusively, advantageous for ankle joints if a position of the first joint part relative to the second joint part can be adjusted and locked in different positions. This renders it possible, for example, to freely adjust an angle between a lower leg section and a foot section of a prosthesis, which in this case forms the first and second joint part. For example, if the wearer of such a prosthesis changes their footwear, it generally also leads to a change in heel height; said adjustability allows this to be taken into account. When using the prosthesis, it is not intended that hydraulic fluid is transferred from one hydraulic chamber to another hydraulic chamber after adjusting the heel height. Such a joint, which does not allow any movement during operation, is highly reliable, thereby giving the wearer of a prosthesis or orthosis equipped with such a joint a sense of security.

Alternative embodiments of hydraulic joints for orthopedic devices provide that the fluid connection is not closed when the joint is in operation. A movement of the two joint parts relative to each other is possible as a result. However, depending on the flow resistance generated by the fluid connection, the movement is damped to a greater or lesser extent. Preferably, it is possible to render the flow resistance and therefore also the damping of the movement adjustable, for example by way of a throttle valve. The prior art includes valve arrangement designs which feature combinations of throttle valve and non-return valves, so that the flow resistances that counter a flow of hydraulic fluid from one hydraulic chamber into another hydraulic chamber can be adjusted individually and preferably differently for different flow directions. Such a joint, with which a damped movement is possible, enables a very natural gait pattern on the one hand and a high level of comfort on the other, as mechanical impacts can be absorbed.

However, it is disadvantageous that a combination of the two effects, of a fixed joint angle on the one hand and a damped movement on the other, seems impossible without a mechatronic control system.

U.S. Pat. No. 9,132,023 B2 discloses an artificial ankle joint that enables a heel-height adjustment on the one hand and dynamic damping on the other. To this end, it has four hydraulic chambers that form two pairs connected to one another. If the joint is to be dynamically damped, the connection between the two chambers of the first pair is closed. The two other chambers are then fluidically connected, so that a movement of the joint displaces hydraulic fluid from one chamber into the other. Damping can then be adjusted via the flow resistance. Conversely, if a heel height is to be adjusted, the closed connection between the chambers of the first pair must be opened and the open connection between the chambers of the second pair closed or it must be ensured in another way that no hydraulic fluid can be exchanged between the chambers of the second pair. The heel height of the first pair can then be adjusted by changing the ratio of the size of the chambers of the first pair. However, it is disadvantageous that the size ratio of the chambers of the second pair is not reproducible and therefore the angle in the ankle joint at which the heel is set cannot be adjusted reproducibly. Moreover, the construction requires a relatively large installation space and is heavy due to the many high-mass elements. This is particularly disadvantageous for ankle joints as they are worn away from the body and have to be accelerated considerably when walking, for example. This causes significant moments of inertia that have to be overcome.

Regardless of the design of the hydraulic system, the problem here is that the two joint parts perform a swivel movement relative to each other about a swivel axis. For an optimal movement of the piston, this rotary movement must be converted into a purely longitudinal movement, i.e. a displacement along one direction. This is technically unproblematic and can be achieved, for example, using a connecting rod. The disadvantage, however, is that this increases the installation height of the hydraulic system and therefore the installation height of the joint, which is a disadvantage in many orthopedic applications and orthopedic devices. In particular when being used for a prosthetic foot or leg prosthesis, the installation space available is very restricted. The hydraulic system is often arranged in the prosthetic foot, which is inserted into a so-called foot shell in order to achieve a visual impression as similar as possible to a human foot.

The invention therefore aims to further develop a joint of the type described above in such a way that the installation height and/or space required can be kept small.

The invention solves the task addressed by way of a joint according to the preamble of claim 1, which is characterized in that the first piston is arranged at a first securing point of the first joint part in such a way that the first piston performs a movement along the longitudinal axis of the first cylinder and a tilting movement when the first joint part is swivelled relative to the second joint part.

The invention is based on the surprising knowledge that it is not necessary for the piston to perform only a longitudinal movement without a change in orientation. Rather, according to the invention provides that this is precisely not the case, but that, in addition to the displacement along the longitudinal axis of the first cylinder, the first piston performs a tilting movement, which alters the orientation of the first piston relative to the first cylinder. Said tilting movement is preferably about a tilt axis, which extends perpendicular to the longitudinal axis of the first cylinder. Particularly preferably, the tilt axis runs parallel to the swivel axis of the joint.

Advantageously, the outer surface of the first piston, which faces an inner surface of the first cylinder, is designed with at least one sealing element, such as an O-ring, preferably made of an elastic material, such as rubber. Particularly preferably, the outer surface of the piston has a groove in which the sealing element is arranged. The sealing element protrudes beyond the outer surface of the first piston. In the non-mounted state, it protrudes beyond the outer surface of the first piston by a distance that is greater than the distance between the outer surface of the first piston and the inner surface of the first cylinder in the mounted state.

Preferably, the bearing between the piston and the first joint part is the only bearing that is moved in a linear manner. All other bearings where two components are connected to each other are rotating bearings. A piston rod is preferably omitted and, particularly preferably, no supporting rings are required. For this purpose, it is advantageous if the kinematics are designed in such a way that the pistons are clearly guided but not mounted so as to be overdetermined. The sealing element is preferably designed in such a way that it seals the piston on which it is arranged against both a round cylinder shape and an elliptical cylinder shape. Such an elliptical seal is required if the piston is tilted relative to the cylinder. The piston is preferably designed as a spherical segment in the area in which the sealing element is arranged. This means that the outer contour of the piston has a constant curvature in this area, i.e. a constant radius about a single central point. To increase the elasticity of the seal created by the sealing element, it is advantageous to deepen a groove, which is arranged in the piston and in which the sealing element is positioned, and to partially fill it with an elastic bearing material. Alternatively or additionally, the sealing element has a sealing lip which, in the unloaded state, protrudes further out of a groove accommodating the sealing element than a ring seal in order to be able to seal a larger sealing gap.

In one preferred embodiment, the first cylinder is arranged in such a way that the path of the first securing point intersects the first longitudinal axis twice when the first joint part swivels relative to the second joint part. The longitudinal axis of the first cylinder is, like the first cylinder, also positioned in the second joint part and cannot be moved relative to said second joint part. The first securing point is part of the first joint part and cannot be moved relative to it. Therefore, if the two joint parts are swivelled relative to each other, the first securing point is also swivelled relative to the first longitudinal axis of the first cylinder. In one preferred embodiment, the first longitudinal axis of the first cylinder extends perpendicular to the swivel axis of the joint, wherein the two axes are not skewed in the mathematical sense, i.e. they do not intersect, even if they are extended in thought.

Given that the first component performs a circular movement or at least part of a circular movement relative to the second component during swivelling, the first securing point also performs a circular movement relative to the first longitudinal axis of the first cylinder. Part of the circular path can either not intersect a straight line, it can touch it once or it can intersect it twice. In this case, the embodiment in which the circular path that describes the securing point during swivelling intersects the first longitudinal axis twice is preferred.

Within the scope of the present invention, this does not necessarily mean that the circular path performed by the first securing point and the first longitudinal axis have to lie within a common plane. While this does constitute an advantageous design, it is not essential for the invention. To achieve the effect described here, it is sufficient for the first longitudinal axis to intersect the vertical projection of the circular path twice. The vertical projection of the circular path is the projection from which the circular path appears to be a circular path.

Given that the two joint parts can only be swivelled relative to each other in a predetermined swivel range, i.e. in a predetermined angular range, the part of the circular path is also limited to this angular range. In a preferred embodiment, the distance of the first securing point from the first longitudinal axis of the first cylinder is the same at both limit angles that delimit the swivel ranges of each device. Particularly preferably, the distance of the first securing point from the first longitudinal axis is the same as in the center position, in which the angle between the first joint part and the second joint part is exactly in the middle of the two limit angles.

The first piston is preferably secured directly at the first securing point of the first joint part. In particular, there is no other moving element between the first joint part and the first piston that can be rotated or tilted about another axis that does not run through the first securing point. In particular, no connecting rod is provided.

Advantageously, the hydraulic system comprises a second cylinder, which is arranged in the second joint part, and a second piston, which is positioned in the second cylinder and arranged at a second securing point of the first joint part. In this case, the second piston is also designed in such a way that, in addition to a movement along the second longitudinal axis of the second cylinder, it performs a tilting movement when the two joint parts swivel relative to each other. Preferably, the first cylinder and the second cylinder, along with the two pistons arranged within them, are designed to be the same, preferably identical.

In one preferred embodiment, the swivel range is delimited by a first limit angle and a second limit angle, wherein the distance between the securing point of the piston and the longitudinal axis of the respective cylinder is the same at both limit angles. Particularly preferably, the distance between the securing point of the piston and the longitudinal axis of the respective cylinder is exactly the same in the middle of the swivel range as at the limit angles. Particularly preferably, the piston is arranged on the joint part such that it can be swivelled about a piston axis.

The hydraulic system preferably has at least one equalizing volume, which is preferably pre-tensioned, especially preferably spring-loaded. Such an equalizing volume is connected to the remaining elements of the hydraulic system in such a way that hydraulic liquid in the hydraulic system can flow into and out of the equalizing volume. This is especially practical when, for example, the volume of the hydraulic liquid changes due to changes in the temperature of the hydraulic liquid. If the volume of the liquid increases, for example because it expands due to an increasing temperature, the excessive volume can flow into the equalizing volume. This preferably occurs against a resistance, for example a force to be overcome that pre-tensions the equalizing volume. This preload can be achieved, for example, in that hydraulic fluid flowing in moves a wall of the equalizing volume or part of such a wall in order to increase the volume available for the hydraulic fluid. The wall or part thereof is preferably displaced, wherein a spring, i.e. an elastic energy store, is charged with potential energy.

Advantageously, at least one sealing element is arranged on the first piston and/or the second cylinder, said sealing element sealing a gap between the first piston and the first cylinder. Particularly preferably, the at least one sealing element is arranged in a groove, wherein the at least one sealing element is preferably mounted on an elastic bearing element, which is arranged at the base of the groove. The at least one sealing element preferably comprises a sealing lip. Irrespective of the specific design of the at least one sealing element, this sealing element is designed in such a way that one hydraulic chamber, which is delimited by the cylinder and the piston, is sealed. This means that no hydraulic liquid can escape between the first cylinder and the first piston at the pressures generated during operation of the hydraulic system. If the hydraulic system also has a second piston and a second cylinder, at least one sealing element is also arranged on the second piston and/or the second cylinder. Preferably, identical sealing elements are arranged. The sealing effect of the at least one sealing element is preferably achieved, regardless of the tilt angles that occur. The width of the gap between the respective piston and the corresponding cylinder varies upon movement of the two joint parts relative to each other. This cannot negatively impact the sealing effect.

Preferably, the joint is characterized in that the hydraulic system has at least one volume, which comprises a first partial volume and a second partial volume, and at least one further fluid line, which comprises a first partial line and a second partial line, wherein the first partial volume is connected to the first hydraulic chamber via the first partial line and the second partial volume is connected to the second hydraulic chamber via the second partial line and that the first partial volume is separated from the second partial volume by a displaceable separating device.

Even if the first partial line and the second partial line are jointly referred to as a further fluid line, this does not mean that they are fluidically connected. Rather, the volume with the first partial volume and the second partial volume, the latter two being separated by the displaceable separating device, is situated between the two partial lines. If fluid is directed out of the first hydraulic chamber, through the first partial line and into the first partial volume, the displaceable separating device must be displaced so as to enlarge the first partial volume. This inevitably leads to a reduction in the second partial volume, so that fluid is directed out of the second partial volume, through the second partial line and into the second hydraulic chamber.

The separating device must therefore be able to change the size of the abutting partial volumes. This can be achieved, for example, by way of a displaceable piston, also referred to as a flying piston. Alternatively or additionally, the separating device has a membrane, which is preferably stretched across the cross-section of the volume. If fluid is now directed into one of the two partial volumes, the membrane bulges due to the increased pressure on the one side, thereby increasing the first partial volume at the expense of the second partial volume, which decreases in the process.

Therefore, even if the fluid connection between the two hydraulic chambers is closed by the valve, the separating device can be moved within the volume in the other fluid line. It is advantageous if the separating device can be moved within the volume, which may be a cylinder for example, but fluid itself cannot pass through the separating device. The separating device preferably rests against the inner wall of the volume. The separating device and volume are preferably designed as a cylinder and a piston adapted to it and can be designed as a longitudinally displaceable system or a rotary hydraulic system. A bent or curved piston is also possible. It is important that a displacement of the piston causes fluid to be displaced from the first hydraulic chamber into the second hydraulic chamber or vice versa. It is therefore sufficient for the further fluid line to comprise a first partial line, which connects the first hydraulic chamber with a first partial volume where the separating device is located, and a second partial line, which connects the second hydraulic chamber with a second partial volume. In this case, the first partial volume and the second partial volume are separated from each other by the separating device.

When the fluid line between the two hydraulic chambers is closed, fluid can be exchanged between the first hydraulic chamber and the first partial volume through the first partial line. It is likewise possible to exchange hydraulic fluid between the second hydraulic chamber and the second partial volume through the second partial line. Since the overall volume, composed of the first partial volume and the second partial volume, remains constant, even when the separating device is moved, a quantity of fluid directed into one of the two partial volumes must be removed from the respective other partial volume.

Preferably, the volume in which the separating device, for example the movably mounted piston, is smaller than the volume of the first hydraulic chamber and smaller than the volume of the second hydraulic chamber. The range of movement of the joint that is also possible when the fluid connection is closed, i.e. when the first partial line and/or the second partial line is closed, between the first hydraulic chamber and the second hydraulic chamber is then small in relation to the range of movement of the joint when the fluid connection is open. If the fluid connection between the first hydraulic chamber and the second hydraulic chamber is closed, fluid can only be moved out of one of the two hydraulic chambers into the volume in the fluid line, where it causes the separating device, for example the moveably mounted piston, to be displaced. On the opposite side of the separating device, this causes fluid to be directed from the volume into the respective other hydraulic chamber. As soon as the separating device can move no further in this direction, the joint can also not be moved any further in this direction.

In one preferred embodiment, this range of movement is restricted by the separating device, such as the piston, within the volume striking an end stop on at least one side, preferably on two sides, when a certain position is reached. From this point onwards, further movement in this direction of movement is no longer possible and the range of movement in this direction of movement restricted. Preferably, at least one of the end stops, particularly preferably both end stops, is equipped with a spring or a damping element, by means of which a movement is possible in a limited range when a sufficiently large force is applied, even if the end stop has already been reached.

The two hydraulic chambers are preferably connected to each other via at least two fluid connections. Valve arrangements are preferably arranged in the two fluid connections, each of the former having a non-return valve, wherein the two non-return valves act in different flow directions. As a result, the flow resistances for different directions can be selected independently from each other. Both fluid connections can preferably be sealed by a valve.

This configuration is advantageous for knee joints, for example. When the valves of the fluid connections are open, a normal flexion and extension of the joint is possible and hydraulic fluid is directed in each case from one hydraulic chamber into the respective other hydraulic chamber via one of the two fluid connections. The flexion resistance and the extension resistance can be adjusted independently of one another via the valve arrangement in the respective fluid connection. When the two valves are closed, a movement of the two joint parts relative to one another is still possible as fluid can be directed from the hydraulic chambers into the partial volumes and vice versa. This enables, for example, a flexing of the knee in the stance phase and a subsequent extension when walking in a plane.

The at least first partial line and/or the second partial line preferably has at least one throttle, by means of which a flow resistance through the first line and/or the second partial line can be adjusted. It is understood that a throttle in the first partial line also changes the flow resistance of the first partial line and a throttle in the second partial line changes the flow resistance of the second partial line. The throttle, which can be designed as a throttle valve for example, is arranged in the first partial line or the second partial line. Of course, it is also possible to use more than one throttle, of which preferably at least one is arranged in the first partial line and at least one in the second partial line.

Advantageously, at least one valve arrangement is arranged in the first partial line and/or the second partial line, by means of which a flow resistance through the first line and/or the second partial line can be adjusted differently for different flow resistances. Such valve arrangements are known in principle from the prior art. They have combinations of throttle valve and non-return valve, which are arranged to act in parallel. The non-return valve ensures that the throttle valve is only passed through in a single flow direction, namely the direction in which the non-return valve prevents flow, and the throttle valve adjusts the desired flow resistance. In one preferred embodiment, the at least one fluid line has two such combinations, wherein the two non-return valves act in opposing directions. The one combination can therefore only be passed through in the first flow direction and the other combination only in the second flow direction.

Preferably, the separating device can be displaced in at least one direction, particularly preferably in two opposite directions, in each case against a spring force applied by a spring element. This also changes the resistance to a displacement of the separating device within the volume and thus also a displacement of the fluid.

Preferably, the movement of the separating device in at least one direction, especially preferably in two opposite directions, is limited is each case by an end stop, which preferably comprises a damping element. The damping element is preferably an elastomer block or a disk spring. Preferably at least one of the end stops, but particularly preferably both end stops, is designed to be adjustable so that a range of movement of the separating device can be adjusted.

In one preferred embodiment, the first hydraulic chamber is separated from the second hydraulic chamber by a main piston, which is arranged and configured such that it can be moved by swivelling the first joint part relative to the second joint part. The term “main piston” serves only to distinguish it from the piston in the volume of the fluid line and does not imply any size or mass ratios. The use of a single main piston enables an especially simple structural design. The first hydraulic chamber and the second hydraulic chamber can be arranged in the same cylinder and in this case are separated from each other by the main piston. The main piston can also be designed to be longitudinally displaceable or in the form of a rotary hydraulic system, in which it performs a rotary movement when displaced.

The first partial line and/or second partial line preferably extend(s) through the main piston. Particularly preferably, the volume in the fluid line, in which the separating device is located, is arranged within the main piston. The entire fluid line is preferably located within the main piston. This results in an increased engineering effort, but reduces the installation space required, which is generally limited in joints for orthopedic devices.

A method for setting a starting position of the first joint part relative to the second joint part of a joint of the type described here preferably comprises the following steps:
positioning the separating device in a predetermined rest position,
opening the fluid connection by activating the valve,
swivelling the first joint part relative to the second joint part until the starting position is reached, and
closing the fluid connection.

Given that the predetermined rest position of the separating device, for example the moveably mounted piston, is assumed before any setting of the starting position, the starting position is easily reproduced. To this end, the separating device, i.e. the piston in the present example, need only be brought into its rest position. For example, if the joint is an ankle joint, the starting position corresponds to a heel height.

The starting position preferably corresponds to a predetermined joint angle between the first joint part and the second joint part.

Preferably, the separating device is displaced within the volume up to an end stop in order to position the separating device in the rest position. To this end, a torque acting about the swivel axis is preferably applied to the first joint part and/or the second joint part. If the predetermined rest position of the separating device is at an end stop, it is especially easy to reach and also easy to adjust for the user of the orthopedic device, such as a prosthesis. The user need only apply a corresponding torque. For example, if the joint is used as an ankle joint of a lower leg prosthesis between a lower leg section and a foot section, the user can, for example, put weight on the forefoot, thereby applying a corresponding torque, causing the separating device to be displaced into its predetermined rest position. Such a torque can also be applied manually. The disadvantage, however, is that the separating device can then only be moved in one direction after setting the starting position, namely away from the end stop.

Alternatively, it is therefore advantageous not to apply a torque acting about the swivel axis to the first joint part and/or the second joint part in order to position the piston in the rest position. To move the separating device in this situation into its predetermined rest position, at least one force must be applied to it that moves it into said rest position. This can be done, for example, by arranging one or more spring elements within the volume in which the piston is movably mounted, which each apply a spring force to it. In a situation in which no external torque and no external forces are acting, said spring elements ensure that the separating device, such as the moveable piston, is brought into its rest position. It is then generally not at an end stop, so that a movement in both directions is possible after setting the starting position.

The spring elements are preferably designed and configured in such a way that they overcome forces and torques exerted and caused by the force of gravity and move the joint into the neutral position, in which the separating device, such as the moveable piston, is in the rest position.

It may be advantageous to use one or multiple spring elements, the spring force of which is enough to move the separating device to one of its end stops and thus reach the rest position.

The invention also solves the addressed task by way of an orthopedic device with a joint of the type described here, which is characterized in that the joint is a hip joint, an ankle joint or a knee joint.

In the following, embodiment examples of the invention will be explained in more detail with the aid of the accompanying drawings. They show:

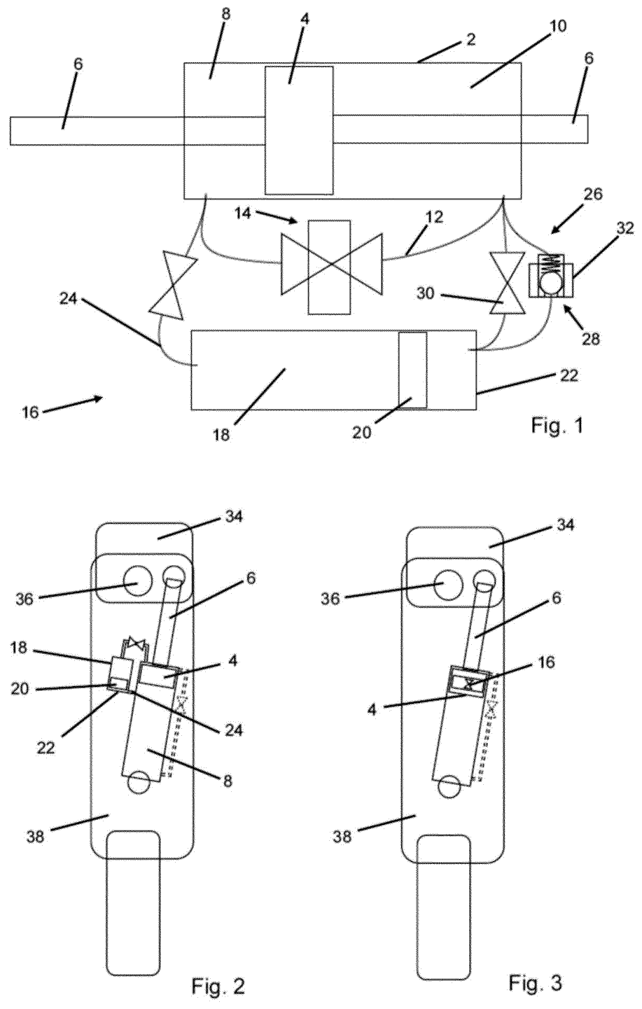

FIG. 1—a schematic circuit diagram of a hydraulic system,

FIGS. 2 to 5—schematic representations of joints,

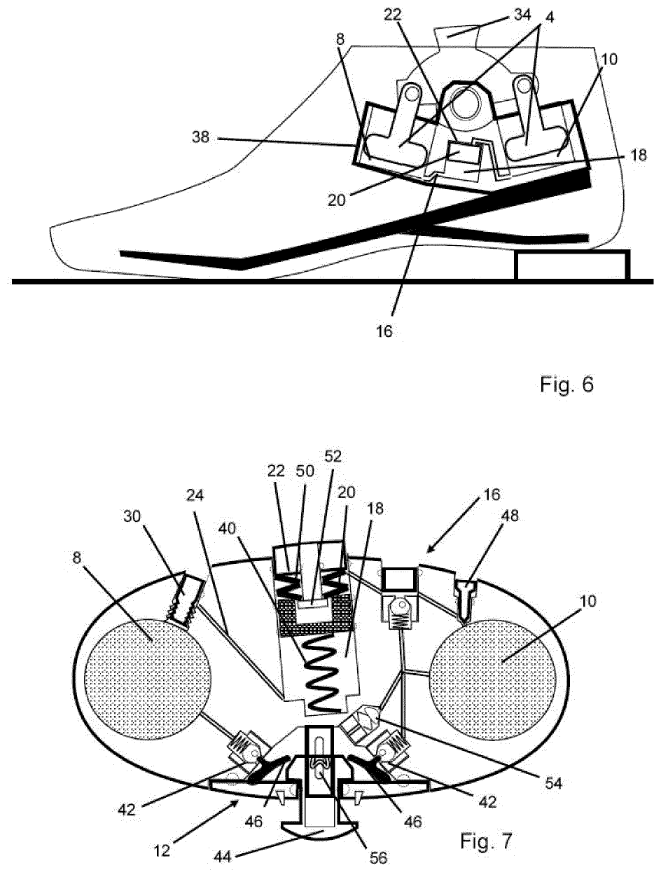

FIG. 6—a schematic representation of a prosthetic foot,

FIG. 7—a schematic top view in a sectional representation,

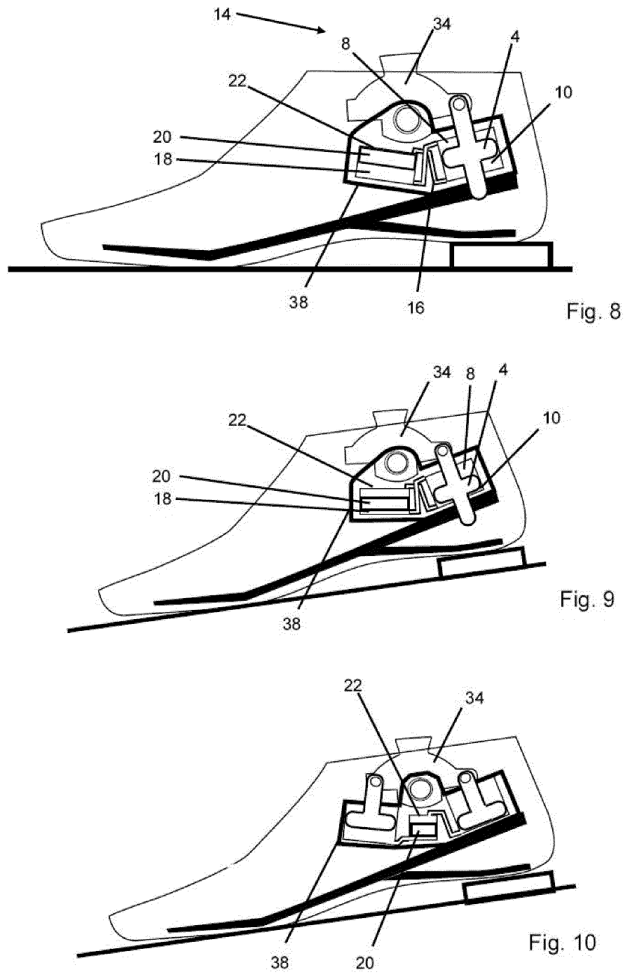

FIGS. 8 and 9—schematic representations of a prosthetic foot according to a further embodiment example of the present invention in two different positions,

FIG. 10—the prosthetic foot from FIG. 6 in a second position,

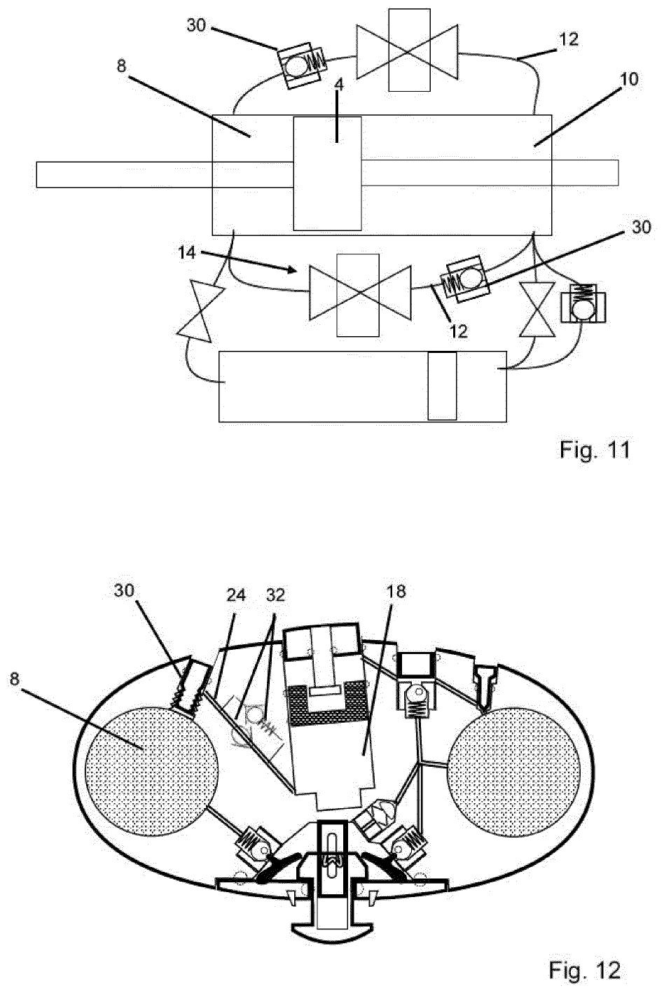

FIG. 11—a different schematic circuit diagram,

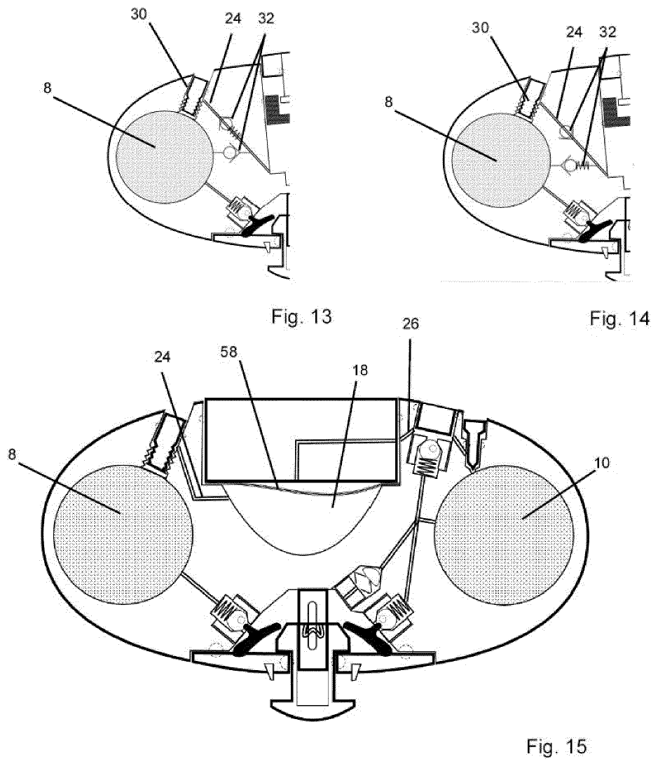

FIGS. 12 to 17—top views of schematic sectional representations of various embodiments,

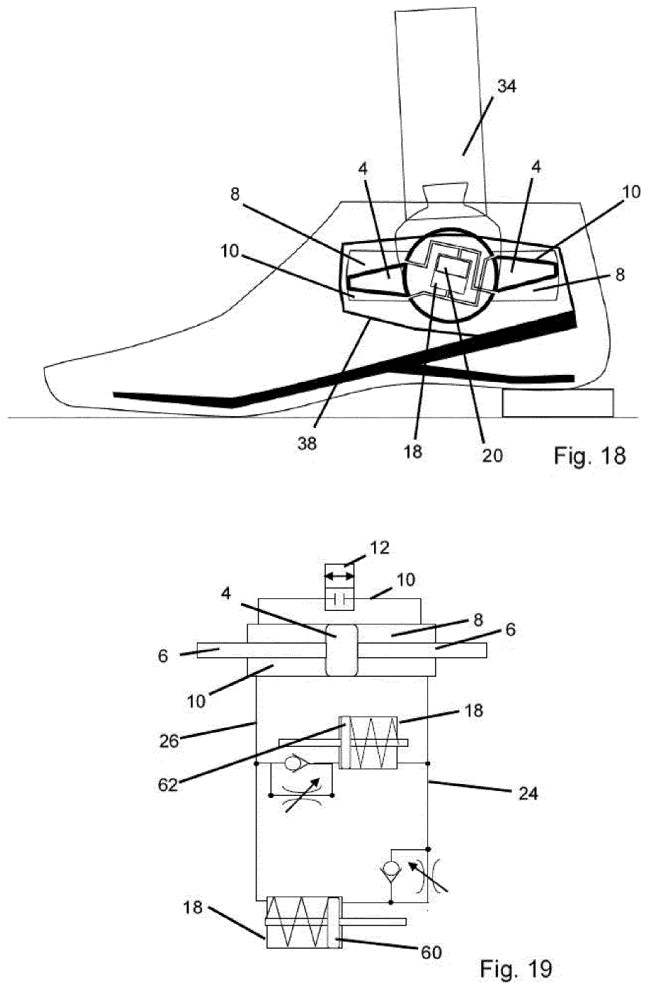

FIG. 18—the schematic representation of a prosthetic foot with a rotary hydraulic system,

FIG. 19—a schematic circuit diagram of a further hydraulic system,

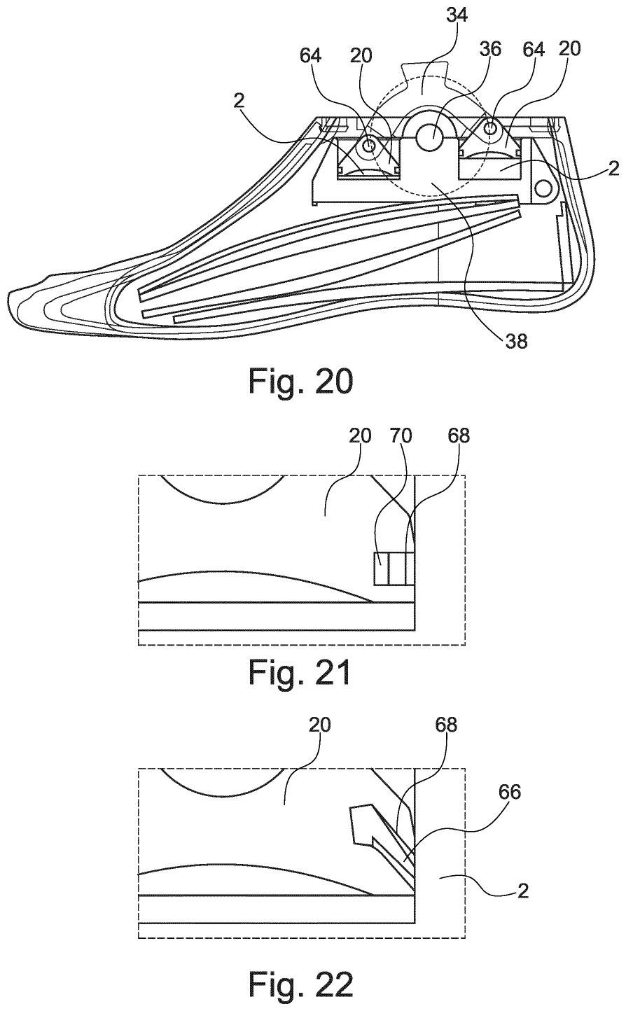

FIG. 20—a schematic representation of a prosthetic foot with a joint according to an embodiment example of the present invention,

FIGS. 21 and 22—detailed views of various sealing elements,

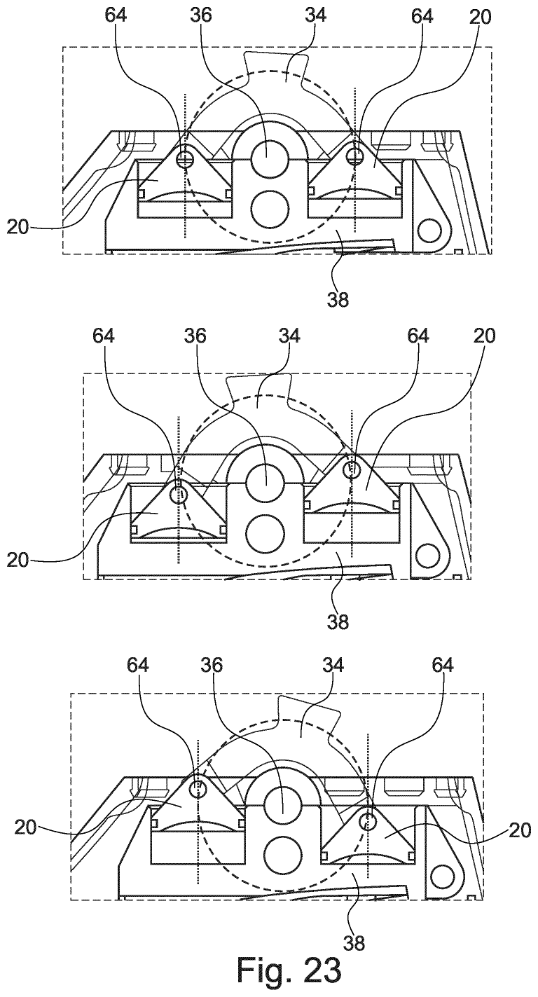

FIG. 23—schematic views of a joint in various swivel positions and

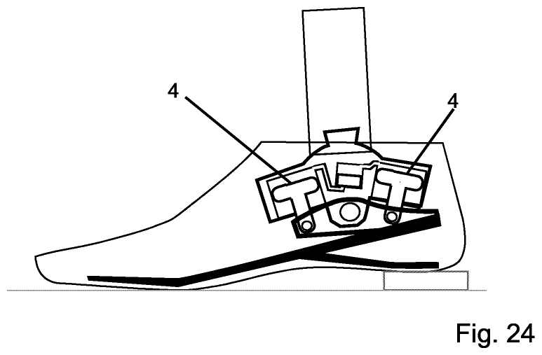

FIG. 24—a schematic representation of a further embodiment.

FIG. 1 shows the schematic representation of a circuit diagram for a hydraulic system of a joint. A main piston 4 is arranged in a cylinder 2, wherein the former can be displaced left and right in the representation shown. It is connected to two piston rods 6 that guide its movement. A first hydraulic chamber 8 and a second hydraulic chamber 10, which are separated from each other by the main piston 4, are located in the cylinder 2. A fluid connection 12 connects the first hydraulic chamber 8 to the second hydraulic chamber 10, a valve 14 being located in the fluid connection 12 that can be opened and closed, meaning that the fluid connection 12 can also be opened and closed. If the fluid connection 12 is opened, hydraulic fluid can flow from the first hydraulic chamber 8 into the second hydraulic chamber 10 and vice versa when the main piston 4 moves. The damping of this movement of the main piston 4 can be adjusted via a potentially adjustable flow resistance caused by the valve 14. If the fluid connection 12 is closed, however, the hydraulic fluid cannot flow through the fluid connection 12.

In addition, the hydraulic system has a further fluid line 16. It comprises multiple elements. It has a volume 18 in which a piston 20 is moveably arranged. This piston 20 can also be moved left and right in the embodiment example shown. However, in this embodiment it does not have a piston rod, but is designed as a flying piston. This is advantageous, but not essential. The piston 20 may also be configured with a piston rod. The piston 20 divides the volume 18 into a first part, which is to the left of the piston 20 in the embodiment example shown, and a second part, which is to the right of the piston 20 in the embodiment example shown. The first part of the volume is connected to the first hydraulic chamber 8 via a first partial line 24. The second part of the volume is connected to the second hydraulic chamber 10 via a second partial line 26. A valve arrangement 28 is located in the second partial line, said arrangement comprising a combination of throttle valve 30 and non-return valve 32. This allows a flow resistance against the fluid flowing through the valve arrangement 28 to be adjusted in a flow direction.

FIG. 2 depicts a joint according to an embodiment example of the present invention as part of a schematically illustrated knee prosthesis. The main piston 4 is arranged with its piston rod 6 on the second joint part 34, which is arranged on a first joint part 38 such that it can be swivelled about a swivel axis 36. In the representation shown, the piston 20 is located at the lower end stop 22 and can therefore only be moved in one direction, upwards in FIG. 2. This occurs when the main piston 4 is moved downwards and fluid moves from the first hydraulic chamber 8 through the first partial line 24 and into the volume 18. This enables a flexing of the joint in the stance phase, for example, which makes walking with the prosthesis more gentle for the wearer and the gait pattern more natural.

FIG. 3 shows a similar embodiment. Here, the piston rod 6 is also coupled with the second joint part 34 of the knee joint, which in turn is connected to the first joint part 38 about the swivel axis 36. However, unlike in FIG. 2 the volume 18 and the entire fluid line 16 is now located within the main piston 4, wherein the fluid line 16 is only schematically depicted for the sake of clarity.

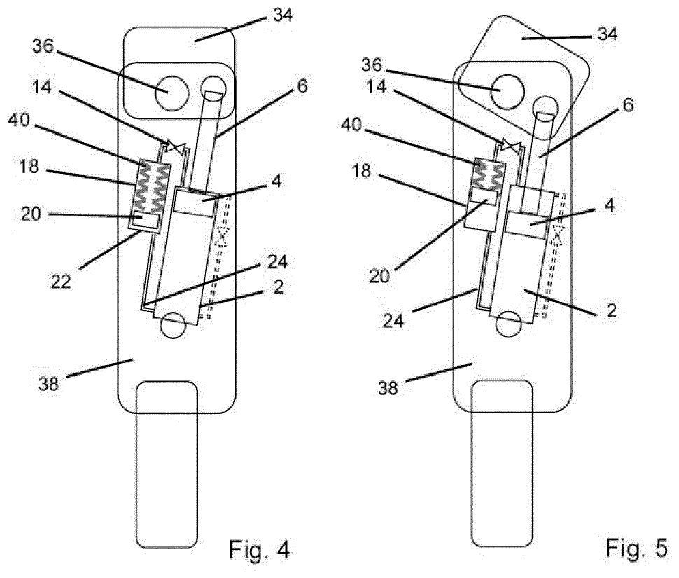

FIGS. 4 and 5 show the identical embodiment. The main piston 4 is in the cylinder 2 and is fixed with the piston rod 6 to the second joint part 34. Unlike in the embodiments in FIGS. 2 and 3, there is a spring element 40 in the volume 18. Said spring element pushes the piston 20 into the rest position at the end stop 22 shown in FIG. 4. Therefore, if the piston 20 is to be moved into its predetermined rest position, it is sufficient to not apply a torque to the first joint part 38 and/or the second joint part 34. The spring element 40 pushes the piston 20 into the rest position. If the second joint part 34 is now swivelled relative to the first joint part 38 about the swivel axis 36, the main piston 4 in the cylinder 2 is moved downwards and pushes fluid through the first partial line 24 into the volume 18, thus moving the piston 20 upwards against the force applied by the spring element 40. This situation is shown in FIG. 5 and denotes flexing in the stance phase.

The connection between the hydraulic chamber and the volume 18 can be closed by way of the valve 14. This renders the movement of the piston 20 impossible.

FIG. 6 depicts a further embodiment example of the present invention as an ankle joint. The first joint part 38 is the prosthetic foot, which is arranged on the second joint part 34 such that it can be swivelled. In the embodiment example shown, the second joint part 34 is configured to be connected to a lower leg element. The piston 4 is designed in the form of two main pistons 4, which form swivel pistons and are each arranged on the second joint part 34 such that they can be swivelled. In each case, the first hydraulic chamber 8 and the second hydraulic chamber 10 are located below the main piston 4. Between the two hydraulic chambers 8, 10 is the fluid line 16, which connects the two hydraulic chambers 8, 10 and in which the volume 18 with the moveable piston 20 is located. In the position depicted in FIG. 6, the moveable piston 20 is positioned at one of its end stops 22, so that a movement of the moveable piston 20 within the volume 18 is only possible in one direction. In FIG. 6 this is a plantar flexion, i.e. a downward movement.

FIG. 7 shows a schematic sectional representation of a top view of the embodiment in FIG. 6. The first hydraulic chamber 8 and the second hydraulic chamber 10 are connected to each other via the fluid connection 12. The valve 14 is designed as a valve arrangement and has two non-return valves 42, each of which can open or close the connection to one of the two hydraulic chambers 8, 10. The arrangement also has a push button 44, which is designed in such a way that, if it is pressed in, i.e. moved upwards in FIG. 7, it actuates the two levers 46 and thus opens the two non-return valves 42. The first partial line 24 is connected to the first hydraulic chamber 8 by a throttle valve 30. A disk spring 50 is depicted at the upper end stop 22, by means of which the end stop 22 is damped. The preload of said disk spring 50 can be adjusted via the adjustable driver 52. The embodiment example shown also depicts a relief valve 54 as well as an opening mechanism 56, by means of which the fluid connection 12 can be opened.

The fluid line 16, composed of multiple partial lines and the volume 18 in the embodiment example shown, is also located between the two hydraulic chambers 8, 10. The piston 20 is located in said volume, the former being pre-tensioned upwards by the spring element 40 in FIG. 7. The spring element 40 is configured to move the piston 20 into its rest position when no other external forces beyond the force of gravity are acting. If the fluid connection 12 is closed, as shown in FIG. 7, a movement of the joint can be achieved by slightly opening the adjustment valve 48. As a result, fluid can flow from the second hydraulic chamber 10 into the volume 18, for example during a heel strike, when increased pressure builds up in the second hydraulic chamber 10, causing the piston 20 to move downwards against the spring force of the spring element 40. A corresponding quantity of fluid flows from the partial volume below the piston 20 into the first hydraulic chamber 8, so that the second joint part 34 moves relative to the first joint part 38.

FIGS. 8 and 9 depict a prosthetic foot similar to the one in FIG. 6. The main difference is that the two hydraulic chambers 8, 10 are separated by a single main piston 4, which is likewise designed as a swivel piston. The two hydraulic chambers 8, 10 are again connected by the fluid line 16 in which the volume 18 with the moveable cylinder 20 is located. As in FIG. 6, the moveable piston 20 is resting against one of its end stops 22 and can therefore only be moved in one direction, downwards in FIG. 8. This position of the moveable piston 30 is preferably assumed when the heel height of the prosthetic foot is determined: in the present embodiment example, essentially the position of the main piston 4 between the hydraulic chambers 8, 10. Once this has happened, the fluid connection 12, which is not shown in FIGS. 6, 8, 9 and 10 and is preferably opened to adjust the heel height, is preferably closed. It is then no longer possible for the fluid to flow from the one hydraulic chamber 8, 10, through the fluid connection 12 and into the respective other hydraulic chamber 10, 8.

FIG. 9 illustrates this situation. Although the fluid connection 12 is closed, the angle between the first joint part 38 and the second joint part 34 has changed compared to the situation in FIG. 8, wherein the main piston 4 has been displaced. As a result, fluid has been displaced from the second hydraulic chamber 10 into the volume 18. In FIG. 9, said fluid is situated above the moveable piston 20 and has moved it downwards. In addition, fluid situated below the moveable piston 20 in FIG. 8 has moved from the volume into the first hydraulic chamber 8.

FIG. 10 shows the situation from FIG. 9 with a prosthetic foot from FIG. 6. The moveable piston 20 moved away from its end stop 22 as the angle between the first joint part 38 and the second joint part 34 changed.

FIG. 11 corresponds to the representation from FIG. 1. The difference, however, is that the first hydraulic chamber 8 and the second hydraulic chamber 10, which are separated from each other by the main piston 4, are no longer connected by one fluid connection 12, but by two fluid connections 12. In both fluid connections 12 there is a valve 14 as well as a throttle valve 30. The valves 14 and/or the throttle valve 30 may be designed differently so as to achieve, for example, different flow resistances for different flow directions of the fluid.

The representations in FIGS. 12 to 17 correspond to the representation in FIG. 7. To avoid repetitions, only the differences shall discussed. Compared to FIG. 7, in FIG. 12 there is a valve arrangement containing two non-return valves 32 in the first partial line 24, which connects the first hydraulic chamber 8 to the volume 18 via the throttle valve 30. Said non-return valves act in different directions, wherein the upper of the two non-return valves 32 in FIG. 12 is spring-loaded. The fluid that flows through this first partial line 24 must pass through the throttle valve 30 regardless of the flow direction.

This is different in FIGS. 13 and 14, each of which depicts a section from a corresponding representation. Again, one of the non-return valves 32 is positioned in the first partial line 24. In the embodiment example shown, this is the spring-loaded non-return valve, which allows fluid to flow from the first hydraulic chamber 8, through the throttle valve 30, through the first partial line 24 and into the volume 18 when the pressure is correspondingly high. Fluid cannot pass through this non-return valve 32 in the opposite direction; rather it passes through the non-spring-loaded non-return valve 32. However, the latter is not arranged in a by-pass in the embodiment example shown, such that the fluid does not have to pass through the throttle valve 30 in this direction.

FIG. 14 illustrates the reversed situation. The non-spring-loaded non-return valve 32, which allows a flow from the volume 18 towards the height of the first hydraulic chamber 8, is positioned in the first partial line 24 in such a way that the fluid flowing through this partial line 24 in this direction passes through the throttle valve 30. The non-return valve 32 acting in the opposite direction, which is spring-loaded, is arranged in the by-pass, so that fluid taking this path does not pass through the throttle valve 30. The skillful selection of the throttle valve and spring of the spring-loaded non-return valve 32 renders it possible to simply and individually adjust the flow resistance for different flow directions.

FIGS. 15 and 17 depict a different embodiment of the present invention. The volume 18 is no longer divided into the two partial volumes by the piston 20, but by a membrane 58. This does not change how it works. Fluid from the first hydraulic chamber 8 can still get into volume 18 below the membrane 58 through the first partial line 24. Fluid from the second hydraulic chamber 10 can get into the second partial volume above the membrane 58 via the second partial line 26. The membrane 58 is designed to be elastic and can thus assume different positions, depending on the prevailing pressure conditions.

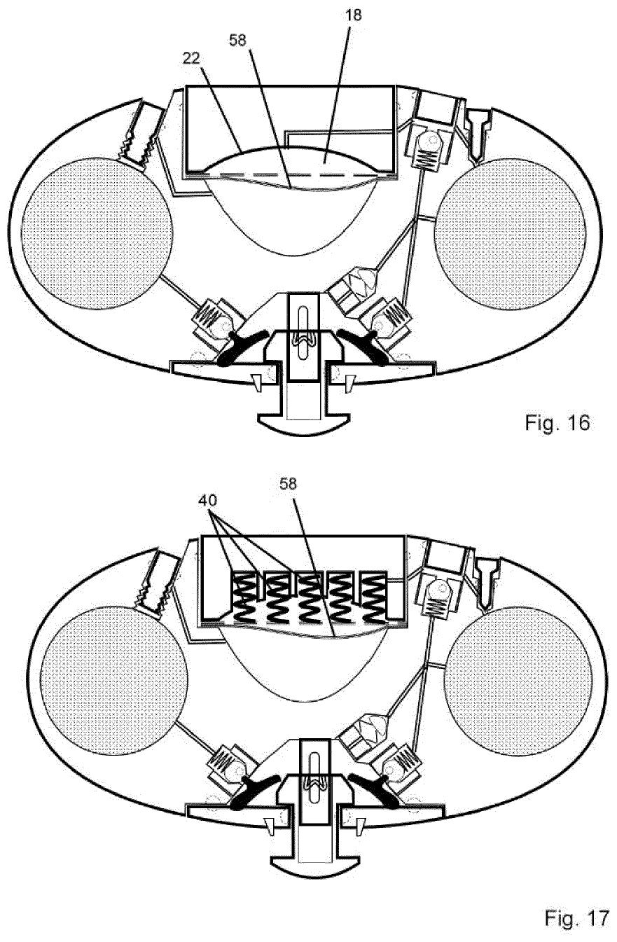

FIGS. 16 and 17 show modified embodiments, each of which however is equipped with the membrane 58. While FIG. 16 only differs from FIG. 15 in that the geometric shape of the volume 18 has been modified, FIG. 17 shows additional spring elements 40. The membrane 58 is preferably designed to be flexible and elastic so that it can rest against the wall delimiting the volume 18 on at least one side. Said wall then acts as an end stop 22 and thus delimits the maximum effective range of the membrane 58. While in this case the end stop 22 in FIG. 16 is designed to be undamped, the embodiment in FIG. 17 is damped by way of the spring elements 40. The membrane 58 initially rests against the lower end of the spring elements 40 in FIG. 17. If further fluid is directed into the first partial volume, shown below the membrane 58 in FIG. 17, the pressure in this area increases, causing the membrane to compress the spring elements, thus enabling a further movement.

FIG. 18 schematically depicts a prosthetic foot with the first joint part 38 and the second joint part 34. The first hydraulic chamber 8 and the second hydraulic chamber 10 are each composed of two parts, which in each case are connected to each other. The prosthetic foot in FIG. 15 has a rotary hydraulic system. The main piston 4 also comprises two parts which are connected to each other in a torque-proof manner. If the joint is moved, the two joint parts 34, 38 are swivelled against each other and the main piston 4 is moved relative to the hydraulic chambers. The parts of the hydraulic chambers 8, 10 upstream of the main piston 4 in the direction of rotation are rendered smaller and the parts of the hydraulic chambers 8, 10 downstream of the main piston 4 in the direction of rotation are enlarged. In FIG. 18, the piston 20 is arranged in the volume 18 between the two parts of the main piston 4 in the area of the axis of rotation of the joint.

FIG. 19 schematically depicts a diagram of a further hydraulic system for a joint for an orthopedic device according to a further embodiment example of the present invention. The main piston 4, which has two piston rods 6 in the embodiment example shown, separates the first hydraulic chamber 8 from the second hydraulic chamber 10. The two hydraulic chambers 8, 10 are connected by the fluid connection 12 where the valve 14 is located. In the embodiment example shown, the volume 18 is composed of two volumes 18. In the first volume is a first displaceable separating device 60 and in the second volume is a second displaceable separating device 62.

If the main piston 4 is displaced to the right in the representation shown, the first hydraulic chamber 8 becomes smaller and part of the fluid contained within is directed through the first partial line 24. The first separating device 60 is displaced to the right as a result. Part of the fluid situated to the right of the first separating device 60 within the corresponding volume 18 is displaced through the second partial line 26 into the second hydraulic chamber 10. Conversely, if the main piston 4 is displaced to the left, the second hydraulic chamber 10 becomes smaller and part of the fluid contained within is directed through the second partial line 26. As a result, the second displaceable separating device 62 is displaced to the right and part of the fluid there is pumped through the first partial line 24 into the first hydraulic chamber 8. The respective flow resistance can be set individually for both directions using the combinations of non-return and throttle valve upstream of the two volumes 18 and the spring elements contained in the volumes 18.

FIG. 20 depicts a prosthetic foot in a schematic sectional view. Spring elements that determine the rolling behavior and elasticity of the foot are depicted schematically only. One recognizes the first joint part 38 in which two cylinders 2 are arranged. The second joint part 34 is arranged thereon such that it can be moved about the swivel axis 36. Two pistons 20 are arranged on the second joint part 34 such that they can be swivelled. If the second joint part 43 is now moved relative to the first joint part 38 about the swivel axis 36, the two pistons 20 in the two cylinders 2 are moved up and down. Given that the securing point 64, at which the respective piston 20 is arranged on the first joint part 38, performs a swivel movement about the swivel axis 36 with the first joint part 38, but the piston 20 can only perform a linear movement in the respective cylinder 2, the pistons 20 are tilted about the securing point 64.

The two pistons 20, of which FIGS. 21 and 22 each depict an enlarged section, each comprise a sealing element 66 that is arranged in a groove 68 provided for this purpose. In FIG. 21, the groove 68 is depicted without a sealing element 66 within it. Instead, a schematically depicted bearing material 70 is located at the base of the groove 68, wherein said bearing material is elastic, thereby enhancing the elastic properties of a sealing element 66 inserted into the groove 68 that is partially filled with the bearing material 70. In FIG. 22, the sealing element 66 is inserted into the groove 68. It has a sealing lip that projects radially outwards from the piston 20, the former sealing the sealing gap between the piston 20 and the cylinder 2.

FIG. 23 shows a joint in three different swivel positions. In the upper representation, the second joint part 34 is in a neutral middle position relative to the first joint part 38. The two securing points 64, at each of which one of the pistons 20 is arranged on the second joint part 34, move on a circular path, depicted by the dashed circular line, when the two joint parts 38, 34 swivel relative to each other. This is the path of the securing points 64 when the two joint parts 38, 34 swivel in relation to each other. Conversely, the pistons 20 move in a straight line, which is depicted by the dotted line. This is the longitudinal axis of the cylinder 2. In the upper representation, the securing points 64 lie on both the circular line and the dotted lines, so that the pistons 20 are not tilted. The sealing line, along which the sealing element seals the hydraulic chambers delimited by the respective piston 20, is therefore a circle.

The middle and lower representations in FIG. 23 depict the second joint part 34 swivelled relative to the first joint part 38. In the middle representation in FIG. 23, the swivelling happens in an anti-clockwise direction. The piston 20 shown on the left has therefore been displaced downwards and the piston 20 on the right upwards. In both representations, the securing points 64 still lie on the dashed circular line as it illustrates the movement of the securing points 64. However, they no longer lie on the vertical dotted lines, which illustrate the movement of the pistons 20. The lower representation in FIG. 23 shows the reverse situation. The two joint parts 38, 34 have been swivelled relative to each other in the clockwise directions so that, in this case, the piston on the left has been displaced upwards and the piston on the right downwards. In these positions of the joint, the sealing line is an ellipsis.

In the neutral position in the upper representation in FIG. 23, the vertical dashed lines extend through the two securing points 64. If the second joint part 34 is swivelled out of this position, both securing points 64 lie between the two dotted lines, so that the respective piston 20 is tilted towards to swivel axis 36. For the piston 20 shown on the left, this means tilting in the clockwise direction. For the piston 20 shown on the right, this means tilting in the anti-clockwise direction.

This only constitutes a possible, albeit very advantageous, embodiment. In a different embodiment, the securing points 64 do not lie on the dashed circular line when in a neutral position, but within the circular line. This means that the pistons 20 assume a tilted position in the neutral position of the joint. Tilting then occurs away from the swivel axis 36. In this embodiment, if the second joint part 34 and the first joint part 38 are swivelled relative to each other, the securing points 64 move until they lie on the dashed lines. The pistons 20 are then no longer tilted and the sealing line is a circle. If the joint is swivelled further in the same direction, the securing points move and lie between the two dotted lines. The pistons 20 are then tilted towards the swivel axis 36.

FIG. 24 shows an embodiment similar to the representation in FIG. 10. The main difference is that the two main pistons 4 are not arranged on the upper joint element, but the lower joint element.

### REFERENCE LIST

2 cylinder
4 main piston
6 piston rod
8 first hydraulic chamber
10 second hydraulic chamber
12 fluid connection
14 valve
16 fluid line
18 volume
20 piston
22 end stop
24 first partial line
26 second partial line
28 valve arrangement
30 throttle valve
32 non-return valve
34 second joint part
36 swivel axis
38 first joint part
40 spring element
42 non-return valve
44 push button
46 lever
48 adjustment valve
50 disc spring
52 driver
54 relief valve
56 opening mechanism
58 membrane
60 first separating device
62 second separating device
64 securing point
66 sealing element
68 groove
70 bearing material

1. A joint for an orthopedic device, comprising:
a first joint part;
a second joint part arranged on the first joint part such that one or more of the first joint part and the second joint part is swivelable about a swivel axis in a swivel range;
a hydraulic system comprising at least a first cylinder which comprises a first longitudinal axis, wherein the at least a first cylinder is arranged in the second joint part, and a first piston is positioned in the at least a first cylinder,
wherein the first piston is arranged at a first securing point of the first joint part such that the first piston is configured to perform a movement along the first longitudinal axis of the at least a first cylinder and a tilting movement upon a relative swivelling movement between the first joint part and the second joint part.

2. The joint according to claim 1, wherein the at least a first cylinder is arranged such that a path of the first securing point intersects the first longitudinal axis twice upon a relative swivelling movement between when the first joint part and the second joint part.

3. The joint according to claim 1 wherein the first piston is directly attached at the first securing point of the first joint part.

4. The joint according to claim 1 wherein the hydraulic system further comprises a second cylinder arranged in the second joint part, and a second piston positioned in the second cylinder, wherein the second piston is arranged at a second securing point of the first joint part, wherein the second cylinder comprises a second longitudinal axis, wherein the second cylinder is arranged such that a path of the second securing point intersects the second longitudinal axis twice upon a relative swivelling movement between when the first joint part and the second joint part.

5. The joint according to claim 4 wherein the swivel range is delimited by a first limit angle and a second limit angle, wherein a distance between the first securing point of the first piston or a distance between the second securing point of the second piston and the longitudinal axis of the respective first or second cylinder is the same at both limit angles.

6. The joint according to claim 5, wherein the distance between the first securing point of the first piston or the distance between the second securing point of the second piston and the longitudinal axis of the respective first or second cylinder in a center of the swivel range is exactly the same as at both limit angles.

7. The joint according to claim 4 wherein at least one piston of the first piston and the second piston is arranged on the first joint part such that it is swivelable about a piston axis.

8. The joint according to claim 1 wherein the hydraulic system comprises at least one equalizing volume.

9. The joint according to claim 1 further comprising at least one sealing element is arranged on the first piston and/or the first cylinder, said at least one sealing element sealing a gap between the first piston and the first cylinder.

10. The joint according to claim 9 wherein the at least one sealing element is arranged in a groove.

11. The joint according to claim 9 wherein the at least one sealing element comprises a sealing lip.

12. The joint according to claim 8 wherein the at least one equalizing volume is pre-tensioned.

13. The joint according to claim 8 wherein the at least one equalizing volume is spring loaded.

14. The joint according to claim 10, wherein the at least one sealing element is mounted on an elastic bearing element which is arranged at a base of the groove.
